(19) United States
(12) Patent Application Publication
(10) Pub. No.: US 2017/0057910 A1
(43) Pub. Date: Mar. 2, 2017

(54) ANTITUMOR NAPHTHALENYL BENZAMIDE DERIVATIVES

(71) Applicant: THE CLEVELAND CLINIC FOUNDATION, Cleveland, OH (US)

(72) Inventors: Frederic J. Reu, Beachwood, OH (US); Sergei Vatolin, Bay Village, OH (US); James G. Phillips, Bay Village, OH (US); Dale Grabowski, Parma, OH (US); Babal Kant Jha, Cleveland, OH (US)

(21) Appl. No.: 15/249,981

(22) Filed: Aug. 29, 2016

Related U.S. Application Data
(60) Provisional application No. 62/210,544, filed on Aug. 27, 2015.

(51) Int. Cl. C07C 233/65

(52) U.S. Cl. CPC C07C 233/65

(57) ABSTRACT

Naphthalenyl benzamide derivatives according to formula I

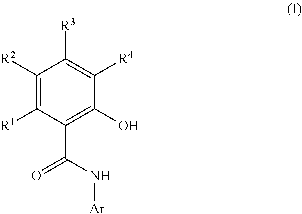

are described, wherein Ar is an aryl or heteroaryl group, and R¹-R⁴ are independently selected from H, halogen, CF₃, hydroxyl, C₁-C₄ alkyl, and C₁-C₄ oxyalkyl, or a pharmaceutically acceptable salt thereof. The naphthalenyl benzamide derivatives can be used for the treatment of cancer in a subject.

### CONTINUING APPLICATION DATA

This application claims the benefit of U.S. Provisional Application Ser. No. 62/210,544, filed Aug. 27, 2015, the disclosure of which is incorporated by reference herein.

### BACKGROUND

Multiple myeloma is an incurable cancer affecting 89,658 people in the US in 2012 and killing 11,240 in 2015 according to 2015 SEER estimates. Most of the deaths from myeloma are due to development of resistance to current therapy, a condition associated with median survival of only about 9 months. Kumar et al., Leukemia 26, 1153 (2012).

When patients have developed treatment resistant myeloma expansion of the transformed clone in the bone marrow and previous exposure to bone marrow toxic therapy has usually reduced normal bone marrow reserve resulting in increased risk of death from infection and bleed. Effective therapy for relapsed/refractory myeloma should therefore not be bone marrow suppressive and remains an unmet clinical need.

Daratumumab, the first anti-CD38 antibody for use in myeloma, gained FDA approval in December 2015 for convincing results in two phase studies that yielded survival over twice of expected survival in patients with refractory myeloma and produced an overall response rate of about 30% as a single agent in refractory myeloma patients. It acts as a CD38 inhibitor and by eliciting an immune response against myeloma, but clinical use is complicated by long infusion times. A small molecule inhibitor of CD38 may overcome this problem, and since CD38 is expressed on other myeloid and lymphoid malignancies, may exert anti-cancer effects in a broader range of cancers.

### SUMMARY OF THE INVENTION

Choosing a mechanistically unbiased approach for identifying normal bone marrow sparing anti-MM compounds with favorable pharmacokinetic activities the inventors identified a small molecule effective against all MM cell lines tested. In vivo study using an established immunocompetent systemic mouse myeloma model (5TGM1-luc) confirmed tolerability and promise as a new drug and therapeutic principle to be developed in MM.

The inventors have developed an assay for multiple myeloma (MM) drug candidates that simultaneously informs about tolerability by normal bone marrow (NLBM), stability towards liver enzymes, and anti-MM activity in the context of cell barriers, bone marrow stromal support, and short, kidney-clearance-like exposure. This assay was used to identify lead compound CCF1172.

CCF1172 had uniform activity against seven genetically heterogenous MM cell lines, and demonstrated in vivo activity in a mouse model of myeloma. Chemical modification revealed that the 2-hydroxy moiety of CCF1172 could not be modified into O-methyl without a complete loss of anti-MM activity using up to 20 μM as dose, indicating the therapeutic principle is based on a salicylic amide backbone rather than on general benzamide chemistry.

In light of this work, the present invention provides naphthalenyl benzamide derivatives according formula I

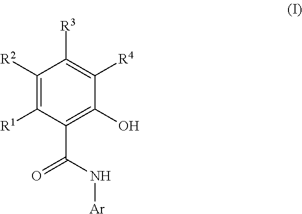

Formula 1 provides a core phenyl amide structure to which is attached a substituted phenyl group (R¹). An Ar group, which is an aryl or heteroaryl group, is also attached through the amide moiety. Additional groups R¹-R⁴ are positioned around the periphery of the phenyl ring, and are independently selected from H, halogen, CF₃, hydroxyl, C₁-C₄ alkyl, and C₁-C₄ oxyalkyl.

### BRIEF DESCRIPTION OF THE FIGURES

FIG. 1A, FIG. 1B, and FIG. 1C provide a scheme showing a sandwich three organ system assay, structures of lead compounds, and a graph of cell viability. Development of the three organ system sandwich assay yields structurally distinct promising anti-myeloma compounds. (a) To address the objective of identifying new clinically translatable treatments for the incurable cancer multiple myeloma (MM) that disrupt critical programs for this malignancy while sparing normal bone marrow, the inventors submitted hits from a mechanistically unbiased primary anti-MM screen to tests that modeled key components of the in vivo situation, initially separately, then in one multi-layered sandwich assay, followed by assessment of anti-MM activity in a panel of genetically heterogeneous MM cell lines. Although CCF1172 was less potent than CCF642 and CCF1118 in the MM cell line panel, it was equally effective in the sandwich assay where it reduced MM1.S-luc luminescence by 70-80% at 15 μM while reducing viable NLBM cells by less than 30%, suggesting it might undergo activation in the liver; (b) Structures of the three lead compounds, CCF642, CCF1118, and CCF1172; and (c) a graph showing the viability of cells 72 hours after CCF642 administration.

FIG. 2A, FIG. 2B, and FIG. 2C provide graphs and images showing CCF1172 activity in a mouse model of myeloma. C57BL/KaLwRij mice were injected with 4×10⁶ 5 TGM1-luc cells per mouse via the tail vein. The next day, treatments were started. Compounds were diluted from DMSO stock into aqueous bovine serum albumin (Sigma, cat. # A7030) solution, supplemented, after compound addition, with NaCl to a final NaCl concentration of 150 mM. In this pilot experiment we aimed to evaluate general tolerance and activity using increasing doses. CCF642 was given in 10% BSA IV twice a week for the first three weeks followed by IP three times a week. IV doses started with 1 mg/kg (3% DMSO) and increased by 1 mg/kg per week (6% DMSO in the second and 10% DMSO in the third week). IP doses started with 6 mg/kg in week 4 and from week 5 onwards used 12 mg/kg (10% DMSO in both). CCF1172 (obtained from SPECS) was given IP three times a week, 22 mg/kg in the first two weeks, then 44 mg/kg (10% BSA, 10% DMSO). No obvious adverse events were observed by three times a week clinical observations and weekly weight measurements. In vivo imaging (a) with average and standard error of the mean displayed in (b) and analysis of time to death or IACUC protocol defined need for euthanasia suggested albumin-based solutions of CCF642 and CCF1172 had anti-MM activity and therefore warranted further study, with (c) providing a graph showing the percentage of live cells after treatment with albumin, albumin-CCF642, and albumin CCF1172.

FIG. 3 provides a hydrophobic surface representation of CCF1172 binding to CD38. CD38 was used for in-silico simulation of CCF-1172 binding to CD38. CCF-1172 binds with a dissociation constant of 0.25 μM. UCSF Chimera was used to analyze the result.

FIG. 4 provides an image showing how CCF-1172 docks into the catalytic site of CD38. CCF-1172 cocks into the catalytic site of CD38 with an adjusted binding energy of dG=8.3 kCal/mol. The side chains of the residue within 3.5 Å of the ligand are shown in as wire.

### DETAILED DESCRIPTION OF THE INVENTION

The present invention provides naphthalenyl benzamide derivatives according to formula I:

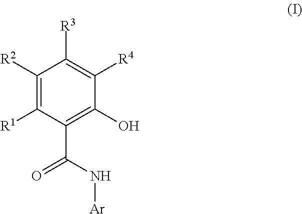

Formula 1 provides a core phenyl amide structure to which is attached a substituted phenyl group (R¹). An Ar group, which is an aryl or heteroaryl group, is also attached through the amide moiety. Additional groups R¹-R⁴ are positioned around the periphery of the phenyl ring, and are independently selected from H, halogen, CF₃, hydroxyl, C₁-C₄ alkyl, and C₁-C₄ oxyalkyl. These naphthalenyl benzamide derivatives are expected to have activity against other cancers and may be used for treatment of cancer in a subject.

### DEFINITIONS

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for naphthalenyl benzamide derivatives are those that do not interfere with the compounds anticancer activity. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Lower alkyl groups are those including at most 6 carbon atoms. Examples of alkyl groups include haloalkyl groups and hydroxyalkyl groups.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. A halo moiety can be chlorine, bromine, fluorine, or iodine.

Cycloalkyl groups are cyclic alkyl groups containing 3, 4, 5, 6, 7 or 8 ring carbon atoms like cyclopropyl, cyclobutyl, cyclopentyl, cyclohexyl or cyclooctyl, which can also be substituted and/or contain 1 or 2 double bounds (unsaturated cycloalkyl groups) like, for example, cyclopentenyl or cyclohexenyl can be bonded via any carbon atom.

A heterocyclyl group means a mono- or bicyclic ring system in which one or more carbon atoms can be replaced by one or more heteroatoms such as, for example, 1, 2 or 3 nitrogen atoms, 1 or 2 oxygen atoms, 1 or 2 sulfur atoms or combinations of different hetero atoms. The heterocyclyl residues can be bound at any positions, for example on the 1-position, 2-position, 3-position, 4-position, 5-position, 6-position, 7-position or 8-position.

The term “aryl” as used herein includes carbocyclic aromatic rings or ring systems. Examples of aryl groups include phenyl, naphthyl, biphenyl, anthracenyl, phenanthracenyl, fluorenyl and indenyl. Aryl groups may be substituted or unsubstituted.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. In accordance with the present invention, desired mechanisms of treatment at the cellular level include, but are not limited to one or more of apoptosis, cell cycle arrest, cellular differentiation, and DNA synthesis arrest.

As used herein, the term “prevention” includes either preventing the onset of a clinically evident unwanted cell proliferation altogether or preventing the onset of a preclinically evident stage of unwanted rapid cell proliferation in individuals at risk. Also intended to be encompassed by this definition is the prevention of metastasis of malignant cells or to arrest or reverse the progression of malignant cells. This includes prophylactic treatment of those having an enhanced risk of developing precancers and cancers. An elevated risk represents an above-average risk that a subject will develop cancer, which can be determined, for example, through family history or the detection of genes causing a predisposition to developing cancer.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of cancer growth by a detectable amount.

The term “subject” for purposes of treatment includes any human or animal subject who has a disorder characterized by unwanted, rapid cell proliferation. Such disorders include, but are not limited to cancers and precancers. For methods of prevention the subject is any human or animal subject, and preferably is a human subject who is at risk of acquiring a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds of the present invention are also useful for veterinary treatment of mammals, including companion animals and farm animals, such as, but not limited to dogs, cats, horses, cows, sheep, and pigs. Preferably, subject means a human.

### Naphthalenyl Benzamide Derivatives

Naphthalenyl benzamide derivatives, as defined herein, include the compounds of formula I:

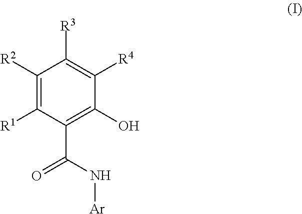

Formula 1 provides a core phenyl amide structure to which is attached a substituted phenyl group (R¹). An Ar group, which is an aryl or heteroaryl group, is also attached through the amide moiety. Additional groups R¹-R⁴ are positioned around the periphery of the phenyl ring, and are independently selected from H, halogen, CF₃, hydroxyl, C₁-C₄ alkyl, and C₁-C₄ oxyalkyl. The naphthalenyl benzamide derivatives also include pharmaceutically acceptable salts of the compounds encompassed by Formula I.

In some embodiments, the Ar group of Formula I is a nitrogen-containing aryl group. A nitrogen-containing aryl group is a heteroaryl group, as defined herein, in which the ring heteroatom is one or more nitrogen atoms. In some embodiments, the nitrogen-containing aryl groups according to formula II, III, or IV:

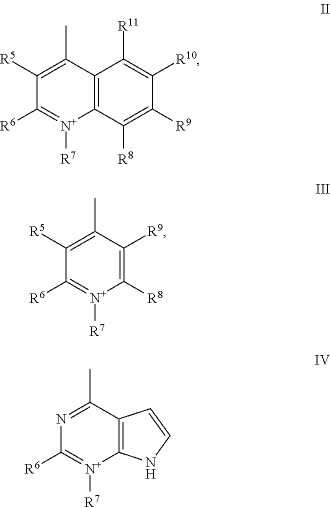

wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

In some embodiments, Ar of formula I is a nitrogen-containing aryl group according to formula II:

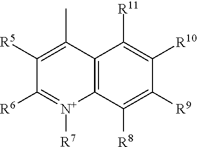

wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

In further embodiments, Ar of the compounds of formula I is a naphthalene group. For example, a preferred compound of formula I is CCF1172 (IUPAC name: 2-chloro-2-hydroxy-N-(napthalene-1-yl)benamide), which has the structure:

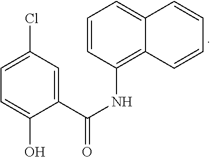 [SMILES: O=C(Nc1cccc2ccccc12)c1cc(Cl)ccc1O]

### Treatment of Cancer Using Naphthalenyl Benzamide Derivatives

The present invention provides methods for treating or preventing cancer in a subject using naphthalenyl benzamide derivatives. A method of treating or cancer in a subject in need thereof by administering a therapeutically effective amount of pharmaceutically acceptable formulation comprising a compound of Formula I:

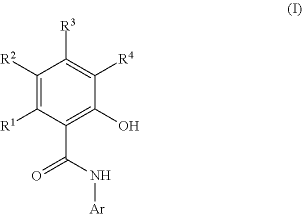

Formula 1 provides a core phenyl amide structure to which is attached a substituted phenyl group (R¹). An Ar group, which is an aryl or heteroaryl group, is also attached through the amide moiety. Additional groups R¹-R⁴ are positioned around the periphery of the phenyl ring, and are independently selected from H, halogen, CF₃, hydroxyl, C₁-C₄ alkyl, and C₁-C₄ oxyalkyl. The naphthalenyl benzamide derivatives also include pharmaceutically acceptable salts of the compounds encompassed by Formula I. The method also encompasses embodiments including the use of any of the subsets of naphthalenyl benzamide derivatives described herein.

Cancer is a disease of abnormal and excessive cell proliferation. Cancer is generally initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. Examples of types of cancer that can be treated using the compounds of the present invention include cancer is selected from the group consisting of leukemia, non-small cell lung cancer, colon cancer, central nervous system cancer, melanoma, ovarian cancer, renal cancer, prostate cancer, and breast cancer. In some embodiments, the cancer is a CD38-expressing cancer.

In some embodiments, the cancer being treated is myeloma or lymphoma. Lymphoma and myeloma are examples of CD38-expressing cancers. Myeloma is a type of cancer that begins in the bone marrow, and is a cancer of B lymphocytes of the plasma. Examples of myeloma include multiple myeloma, which is by far the most common type of melanoma; plasmacytoma, in which only one site (e.g., tumor) of myeloma cells evident in the body; localized myeloma, which is found in one site with exposure to neighboring sites; and extramedullary myeloma, in which the melanoma occurs in a tissue other than the marrow, such as the skin, muscles or lungs.

As noted, the most common type of myeloma is multiple myeloma. Multiple myeloma develops in B lymphocytes after they have left the part of the lymph node known as the germinal center. The normal cell line most closely associated with multiple myeloma cells is generally taken to be either an activated memory B cell or the precursor to plasma cells, the plasmablast. Multiple myeloma is diagnosed based on blood or urine tests finding abnormal antibodies, bone marrow biopsy finding cancerous plasma cells, and medical imaging finding bone lesions. Another common finding is high blood calcium levels. However, because many organs can be affected by multiple myeloma, the symptoms and signs vary greatly.

Lymphoma is a group of blood cell tumors that develop from lymphatic cells. The two main categories of lymphomas are Hodgkin lymphomas and the non-Hodgkin lymphomas. About half of cases of Hodgkin's lymphoma are due to Epstein-Barr virus infection. The main symptom of lymphoma is lymphadenopathy, which is a swelling of the lymph nodes, and lymphoma is definitively diagnosed by detecting Hodgkin's cells such as multinucleated Reed-Sternberg cells using a lymph node biopsy.

The naphthalenyl benzamide derivatives of the invention can be used for both prophylactic and therapeutic treatment. The naphthalenyl benzamide derivatives can, for example, be administered prophylactically to a mammal prior to the development of cancer. Prophylactic administration, also referred to as prevention, is effective to decrease the likelihood that cancer will develop in the subject. Alternatively, naphthalenyl benzamide derivatives of the invention can, for example, be administered therapeutically to a subject that already has cancer. In one embodiment of therapeutic administration, administration of the naphthalenyl benzamide derivatives is effective to eliminate the cancer; in another embodiment, administration of the naphthalenyl benzamide derivatives is effective to decrease the symptoms or spread of the cancer.

The effectiveness of cancer treatment may be measured by evaluating a reduction in tumor load or decrease in tumor growth in a subject in response to the administration of the naphthalenyl benzamide derivative. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof. In some embodiments, candidate anticancer agents can be detected using an in vitro assay system, such as the test developed by Reu et al., described in U.S. Pat. No. 9,389,220, which is incorporated by reference herein, in which liver cells and bone marrow cells are used to evaluate various characteristics of candidate anticancer agents.

Methods of cancer treatment using the compounds described herein can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, and administration of immunotoxins.

### Administration and Formulation of Naphthalenyl Benzamide Derivatives

The present invention also provides pharmaceutical compositions that include naphthalenyl benzamide derivatives according to formula I as an active ingredient, and a pharmaceutically acceptable liquid or solid carrier or carriers, in combination with the active ingredient. Any of the compounds described above as being suitable for the treatment of cancer can be included in pharmaceutical compositions of the invention.

The naphthalenyl benzamide derivatives can be administered as pharmaceutically acceptable salts. Pharmaceutically acceptable salt refers to the relatively non-toxic, inorganic and organic acid addition salts of the naphthalenyl benzamide derivatives. These salts can be prepared in situ during the final isolation and purification of the naphthalenyl benzamide derivatives, or by separately reacting a purified naphthalenyl benzamide derivative with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions includes one or more naphthalenyl benzamide derivatives together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, albumin, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The naphthalenyl benzamide derivatives can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing the naphthalenyl benzamide derivatives, or as a solution or suspension in an aqueous liquor or non-aqueous liquid such as a syrup, an elixir, an emulsion, or a draught. Such compositions and preparations typically contain at least about 0.1 wt-% of the active agent. The amount of protein naphthalenyl benzamide inhibitor (i.e., active agent) is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

### Preparation of the Compounds

Compounds of the invention may be synthesized by synthetic routes that include processes similar to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### A Novel Three Organ System In Vitro Assay Identifies New Protein Disulfide Isomerase Inhibitor with In Vivo Activity Against Myeloma

Cell lines: Multiple myeloma cell lines MM1.S, MM1.R, NCI-H929 were obtained from ATCC. MM cell lines KMS-12-PE and KMS-12-BM were from the JCRB, JJN-3 from DSMZ, and 5TGM1-luc cells were from Dr. Yoneda at the University of Texas Health Science at San Antonio. HRMM.09 cells were generated in the inventor's lab from a patient who developed refractory myeloma after treatment with steroids, IMiD, proteasome inhibitors, DNA alkylators, and anthracyclines. MM1.S-luc and HRMM.09-luc were generated in the inventor's laboratory by transduction with Cignal™ firefly luciferase control lentivirus obtained from Qiagen™ Lymphoma cells were generated by Mitchell Smith. All MM and lymphoma cells were grown in RPMI 1640 (NaCl 103.45 mM, NaCO₃ 23.81 mM, Na₂HPO₄ 5.63 mM, KCl 5.33 mM, Ca(NO₃)₂ 4H₂O 0.424 mM, MgSO4 0.407 mM, pH around 7.2), supplemented with 10% fetal bovine serum, penicillin G (50 units ml⁻¹), and streptomycin (50 μM ml⁻¹). HS-5 cells were grown in appropriate cell culture media. Normal bone marrow cells were obtained from discarded bags of bone marrow grafts from healthy donors and either kept in RPMI with 25% (v/v) HS-5 supernatant for up to 14 days for isolated cytotoxicity assays or frozen and thawed for the sandwich assay. All cells were cultured at 37° C., 5% CO₂ in humidified air.

Reagents: The small molecule library was obtained from SPECS, recombinant proteins were from Enzo life sciences and Biovision, di-eGSSG was from Cayman chemical. Antibodies were purchased from CellSignaling.

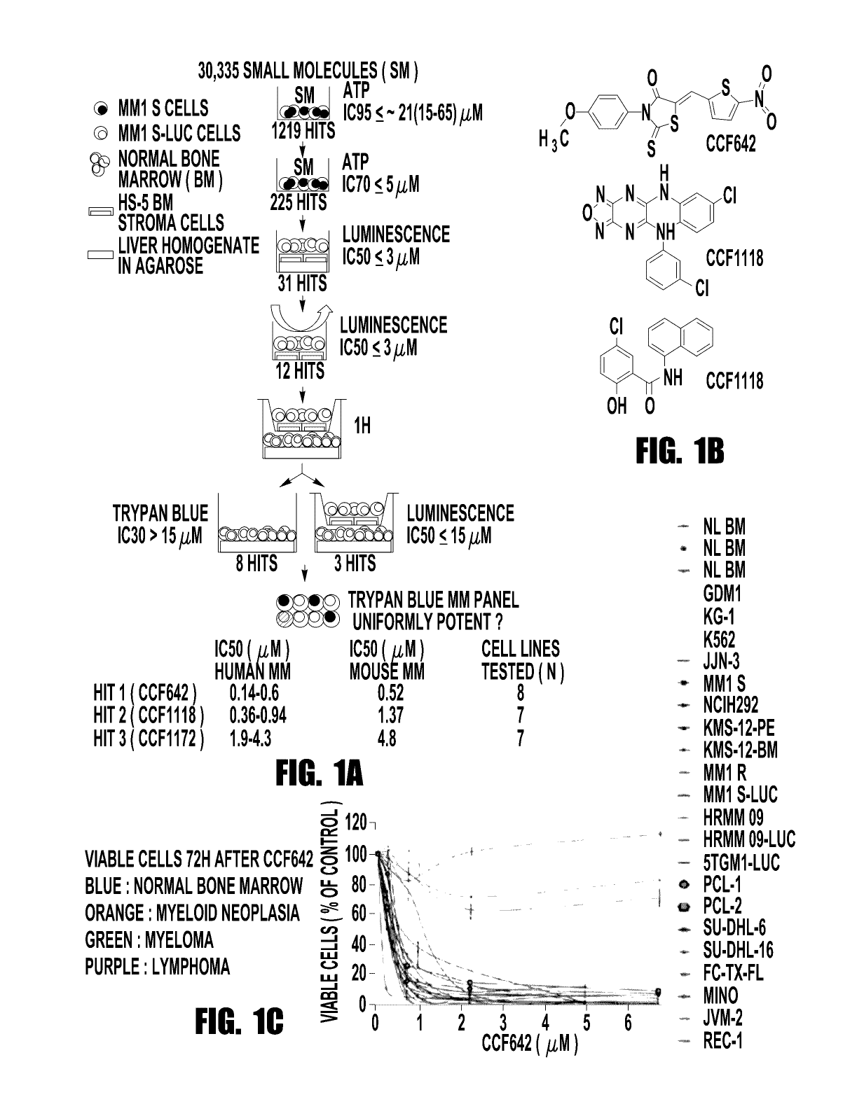

Sandwich assay: HS-5 bone marrow stromal cells which secrete hematopoietic growth factors including multiple myeloma (MM) supporting IL-6 are grown to confluence on Transwell™ inserts before firefly luciferase expressing MM1.S cells are added and incubated overnight. The next day drug candidate is mixed with liver homogenate in separate plates and incubated for 30 min at 37° C. before low melting liquid agarose (cooled to 35-40° C.) is added and solidified at room temperature. Now, RPMI media containing 25% HS-5 supernatant and normal bone marrow (NL BM) cells, obtained from discarded bags of healthy bone marrow donors and freshly thawed, are added and HS-5/MM1.S-luc cell containing inserts are placed on top for 1 h (+/− as desired), then placed into drug free wells simulating kidney clearance. Three to four days later MM luciferase activity is measured and viability of NL BM, constantly drug exposed for maximal stringency, is assessed by trypan blue exclusion using Vicell™ flow. The sandwich assay is shown in FIG. 1.

PDI activity assay: The di-eosin-di-gluthathione assay was performed essentially as described (Raturi, A. and Mutus, B Free Radical Biology & Medicine 43; 2007) with the minor modification that we used gluthathione instead of DTT as reducing agent. Briefly, recombinant PDI was incubated with CCF1172 or analogs in potassium-based assay buffer at 37° C. before reactions were started by addition of gluthathione and di-eosin-di-gluthathione. Release of eosin was measured every 60 seconds for 30 min using excitation/emission filters of 525 nm/545 nm, respectively.

### CCF1172 Discovery

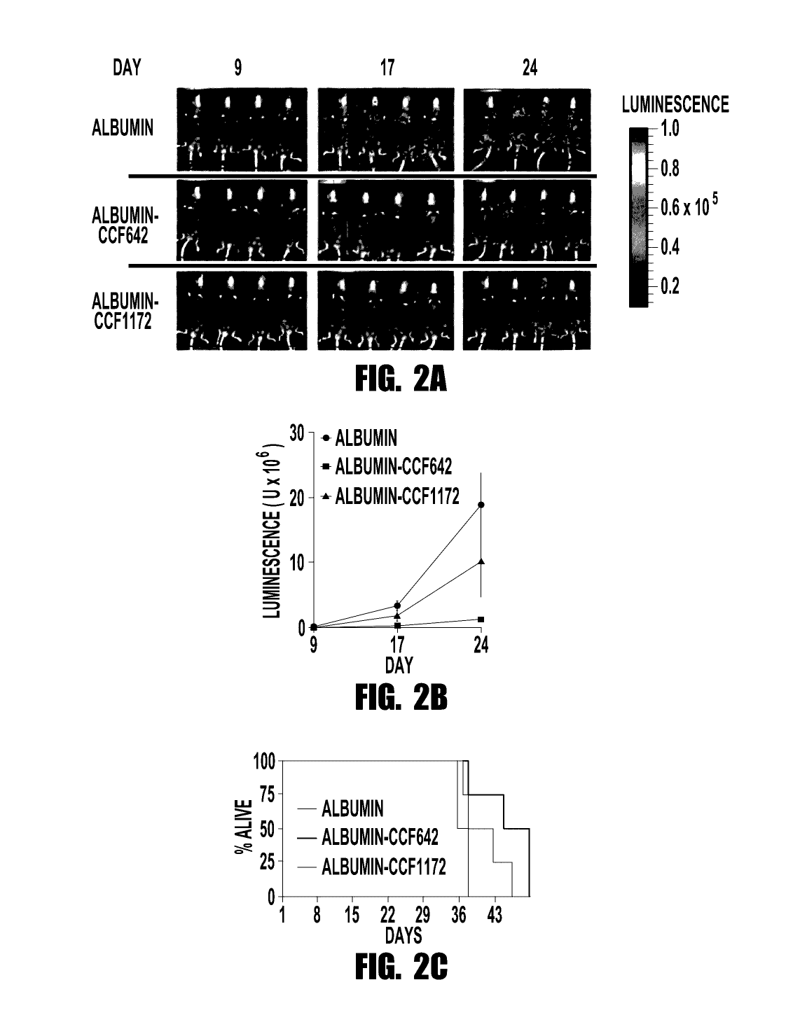

Taking a mechanistically unbiased approach to drug discovery the inventors developed a sandwich assay that selected anti-MM compounds from a primary ATP-based 30,000 small molecule screen for lack of toxicity on normal bone marrow, stability towards liver enzymes, and activity in the context of cell barriers, bone marrow stromal support, and short, kidney-clearance-like exposure. CCF1172 emerged as one of three promising compounds and had sub-μM IC₅₀ potency in all 8 MM cells tested and 6 of 7 lymphoma cell lines. See FIG. 2.

### Example 2

### Preparation of CCF1172 and a CCF1172 Derivative

Procedures for preparing CCF1172 and a CCF1172 derivative are provided below.

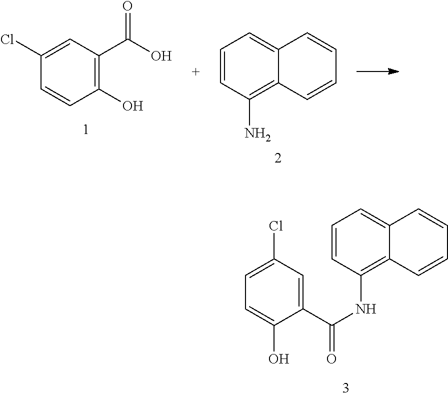 [SMILES: O=C(O)c1cc(Cl)ccc1O ; Nc1cccc2ccccc12 ; O=C(Nc1cccc2ccccc12)c1cc(Cl)ccc1O]

Procedure: To a DMF (16 ml) solution of 1-Aminonaphthalene (0.83 g, 5.80 mmoles), 5-chlorosalicyclic acid (1.0 g, 5.79 mmoles), and triethylamine (1.17 g, 11.58 mmoles) at rt was added HATU (2.42 g, 6.36 mmoles). After stirring for 5 hours, saturated sodium bicarbonate (50 ml) and dichloromethane (70 ml) were added. The organic layer was separated, washed 3× with saturated sodium chloride solution (50 ml) and 1× with 1N HCl solution (30 ml), and then dried over MgSO₄, filtered, and concentrated. Purification by flash silica gel chromatography (Buchi 40 g flash column) using CH₂Cl₂/Hexanes gave a light green solid. Trituration with approximately 30 ml of CH₂Cl₂/Hexanes (2:8) and collection and drying of the solid gave 302 mgs of 3.

¹H NMR (400 MHz, CDCl₃₊ 1 drop of DMSOd6): δ 11.90 (s, 1H), 10.36 (s, 1H), 8.23 (d, 1H), 7.9 (m, 1H), 7.78 (m, 1H), 7.72 (d, 1H), 7.68 (d, 1H), 7.4 (m, 3H), 7.25 (m, 1H), 6.88 (d, 1H).

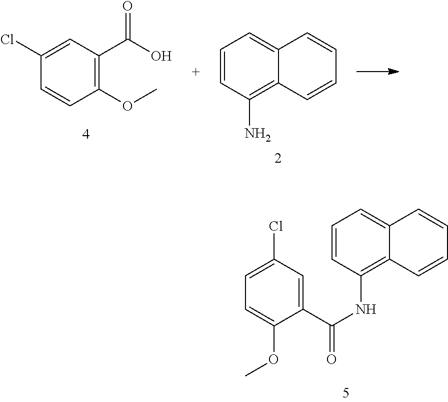 [SMILES: COc1ccc(Cl)cc1C(=O)O ; Nc1cccc2ccccc12 ; COc1ccc(Cl)cc1C(=O)Nc1cccc2ccccc12]

Procedure: To a DMF (40 ml) solution of 1-Aminonaphthalene (2.3 g, 16.06 mmoles), 5-chloro-2-methoxybenzoic acid (3.0 g, 16.09 mmoles), and triethylamine (4 g, 39.52 mmoles) at rt was added HATU (6.11 g, 16.06 mmoles). After stirring for 15 hours, saturated sodium bicarbonate (50 ml) and dichloromethane (70 ml) were added. The organic layer was separated, washed 3× with saturated sodium chloride solution (50 ml) and 1× with 1N HCl solution (30 ml), and then dried over MgSO₄, filtered, and concentrated. Trituration with approximately 6×10 ml of ethyl acetate/Hexanes (2:8) and collection and drying of the solid gave 2.5 grams of 5.

¹H NMR (400 MHz, CDCl₃): δ 10.23 (s, 1H), 8.32 (d, 1H), 8.28 (d, 1H), 7.9 (d, 1H), 7.87 (d, 1H), 7.67 (d, 1H), 7.50 (m, 3H), 7.43 (m, 1H), 7.0 (d, 1H), 4.15 (s, 3H).

### Example 3

### Computational Modeling Studies

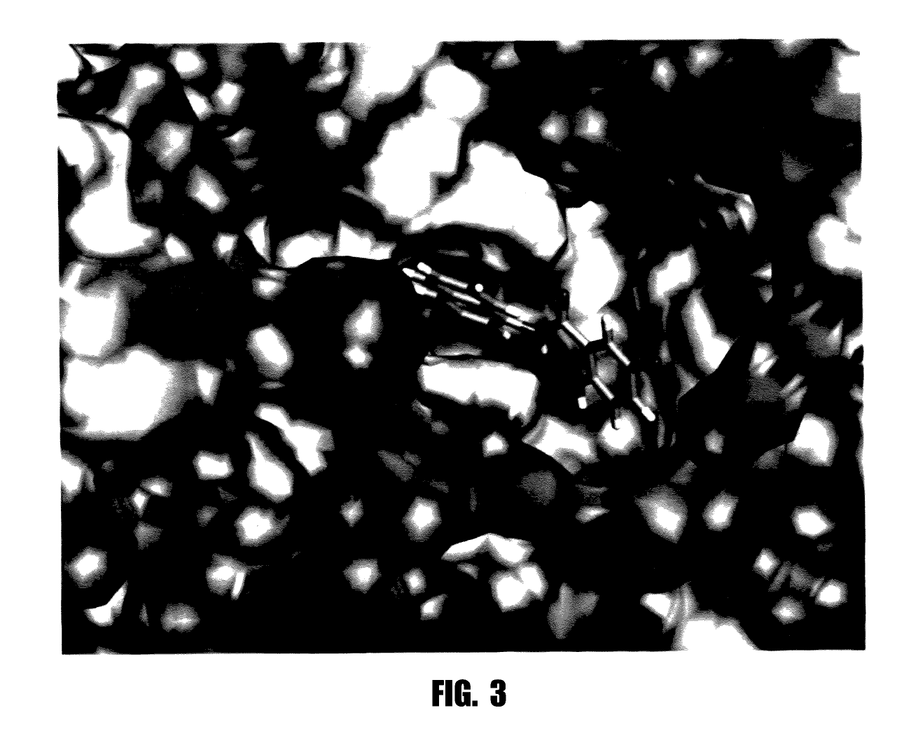

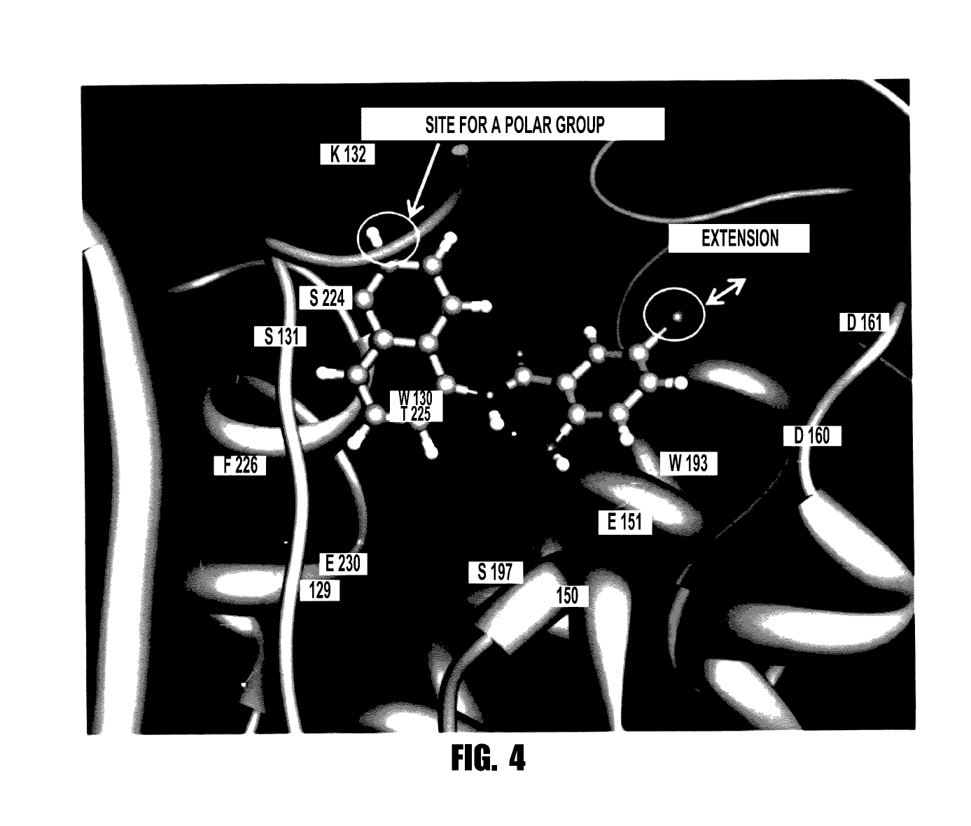

Computational modeling results are based on screening nearly 1,000 high resolution 3D structures of different proteins in the RCSB protein data bank in-silico using the small molecule drug suite (Schrodinger Inc.), and are shown in FIGS. 3 and 4. A hierarchical series of filters were used for all possible locations of ligands in the active site region of target molecules. CCF-1172 binds with significantly high scoring function in the active site of CD38. The final pose was established by energy minimization using OPLS3. The figures were generated in Chimera 1.8. CD38 is expressed at high density on myeloma cells and can currently only be targeted with anitocies clinically.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A compound of formula I 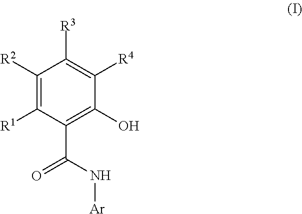
wherein Ar is an aryl or heteroaryl group, and
R¹-R⁴ are independently selected from H, halogen, CF₃, hydroxyl, C₁-C₄ alkyl, and C₁-C₄ oxyalkyl, or a pharmaceutically acceptable salt thereof.

2. The compound of claim 1, wherein Ar is a nitrogen-containing aryl group.

3. The compound of claim 2, wherein Ar is selected from nitrogen-containing aryl groups according to formula II, III, or IV: 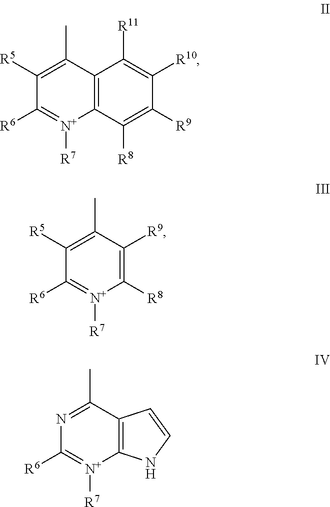
wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

4. The compound of claim 1, wherein Ar is according to formula II: 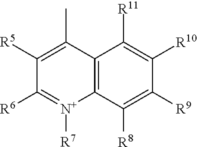
wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

5. The compound of claim 1, wherein Ar is a naphthalene group.

6. The compound of claim 5, wherein the compound is

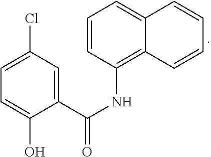 [SMILES: O=C(Nc1cccc2ccccc12)c1cc(Cl)ccc1O]

7. A method of treating cancer in a subject in need thereof by administering a therapeutically effective amount of a compound of Formula I: 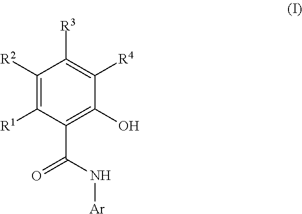
wherein Ar is an aryl or heteroaryl group, and
R¹-R⁴ are independently selected from H, halogen, CF₃, hydroxyl, C₁-C₄ alkyl, and C₁-C₄ oxyalkyl, or a pharmaceutically acceptable salt thereof.

8. The method of claim 7, wherein Ar is a nitrogen-containing aryl group.

9. The method of claim 8, wherein Ar is selected from nitrogen-containing aryl groups according to formula II, III, or IV: 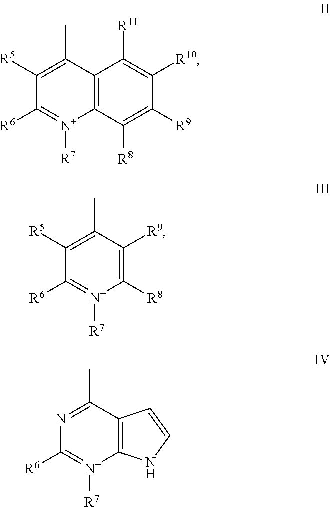
wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

10. The method of claim 7, wherein Ar is according to formula II: 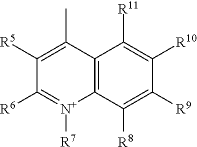
wherein R⁵, R⁶ and R⁸-R¹¹ are independently selected from H, halogen, CF₃, hydroxyl, C₁-C₄ alkyl, and C₁-C₄ oxyalkyl and R⁷ is H or a quaternary salt.

11. The method of claim 7, wherein Ar is a naphthalene group.

12. The method of claim 11, wherein the compound is

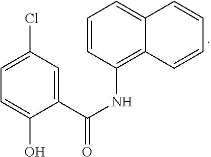 [SMILES: O=C(Nc1cccc2ccccc12)c1cc(Cl)ccc1O]

13. The method of claim 7, wherein the compound of Formula I is administered together with a pharmaceutically acceptable carrier.

14. The method of claim 7, wherein the cancer is myeloma or lymphoma.

15. The method of claim 7, wherein the cancer is multiple myeloma.

16. The method of claim 7, wherein the subject is human.
